(19) United States
(12) Patent Application Publication
(10) Pub. No.: US 2017/0100598 A1
(43) Pub. Date: Apr. 13, 2017

(54) IMPLANT ILLUMINATION

(71) Applicant: GLUSENSE LTD., Rehovot (IL)

(72) Inventors: Yossi GROSS, Moshav Mazor (IL); Boaz BRILL, Rehovot (IL)

(73) Assignee: GLUSENSE LTD., Rehovot (IL)

(21) Appl. No.: 14/881,431

(22) Filed: Oct. 13, 2015

(51) Int. Cl. A61N 1/378; A61M 39/02; H02J 7/02

(52) U.S. Cl. CPC A61N 1/3787; H02J 7/025; A61M 39/0208; A61M 2039/0238

(57) ABSTRACT

An implant system includes an implant, which is configured to be implanted in a body of a subject, and an external controller. The external controller includes a wireless energy transmitter. The implant comprises a wireless energy receiver, which is configured to receive energy from the wireless energy transmitter; and one or more electrical light sources, which are (a) electrically coupled to the wireless energy receiver, and (b) configured to emit light upon being triggered by the wireless energy receiver receiving the energy from the wireless energy transmitter.

### FIELD OF THE APPLICATION

The present invention relates generally to medical devices, and specifically to implantable medical devices.

### BACKGROUND OF THE APPLICATION

Some medical implants function using energy transmitted through the skin by an external charger. Some medical implants deliver a drug from a drug reservoir of the implant.

### SUMMARY OF THE APPLICATION

In embodiments of the present invention, an implant system comprises an implant and an external controller. The external controller comprises a wireless energy transmitter, and the implant comprises a wireless energy receiver, which is configured to receive energy from the wireless energy transmitter. The implant further comprises one or more electrical light sources, which are electrically coupled to the wireless energy receiver, and are typically configured to emit visible light upon being triggered by the wireless energy receiver receiving the energy from the wireless energy transmitter. The one or more light sources are typically arranged so as to indicate a target location on the implant. A user places the external controller near an external surface of skin of the subject. The user places the external controller in a vicinity of the implant, by moving the external controller until the one or more light sources begin to emit the visible light. Typically, the user then positions the external controller directly over, or nearly directly over, the target location by moving the external controller responsively to the visible light emitted by the one or more light sources. Such accurate positioning may, for example, aid in transmitting energy from the external controller to the implant, or in enabling the user to fill a reservoir, such as a drug reservoir, of the implant from the external controller.

For some applications, the one or more light sources comprise a plurality of light sources, for example, between two and four light sources, or at least three light sources. For some applications, in order to aid the user's identification of the target location on the implant, the at least three light sources are disposed at vertices of a regular polygon, such as a triangle or a square, and/or are arranged around the target location.

For some applications, the implant comprises an electrical tissue stimulator. For example, the electrical tissue stimulator may be configured to apply electrical stimulation to nervous tissue, cardiac tissue, and/or muscle tissue. For some of these applications, the electrical tissue stimulator is electrically coupled to the wireless energy receiver, and is configured to apply electrical stimulation to tissue of the subject when powered by the wireless energy receiver.

For some applications, the implant comprises a reservoir, such as a drug reservoir, and an external surface of the implant is shaped so as to define a fluid port, which is in fluid communication with the reservoir. The one or more light sources guide the user to move the external controller toward the fluid port, in order to transdermally fill the reservoir. Typically, the reservoir is a first reservoir, and the external controller comprises a second reservoir. For some applications, the external controller further comprises a needle, which is sized for insertion into the fluid port. After positioning the needle over the fluid port at the target location, the user inserts the needle of the external controller through the skin and into the fluid port, and typically uses the external controller to inject a liquid, such as a drug, through the needle and the fluid port, and into the first reservoir.

There is therefore provided, in accordance with an application of the present invention, apparatus including:

an external controller, which includes a wireless energy transmitter; and

an implant, which is configured to be implanted in a body of a subject, and which includes:
a wireless energy receiver, which is configured to receive energy from the wireless energy transmitter; and
one or more electrical light sources, which are (a) electrically coupled to the wireless energy receiver, and (b) configured to emit light upon being triggered by the wireless energy receiver receiving the energy from the wireless energy transmitter.

For some applications, the one or more electrical light sources are arranged such that at least 10% of the light emitted from the one or more electrical light sources is emitted from an external surface of the implant.

For some applications, the one or more electrical light sources are arranged such that at least 25% of the light emitted from the one or more electrical light sources is emitted from the external surface of the implant.

For some applications, the one or more electrical light sources are arranged so as to indicate a location of a target location on the implant.

For some applications, the wireless energy receiver coincides with or overlaps the target location.

For some applications, the one or more electrical light sources include a plurality of electrical light sources.

For some applications, the plurality of electrical light sources includes between two and four electrical light sources.

For some applications, the plurality of electrical light sources includes at least three electrical light sources.

For some applications, the at least three electrical light sources are disposed at vertices of a regular polygon.

For some applications, the at least three electrical light sources are arranged around a target location on the implant.

For some applications, the at least three electrical light sources are arranged equidistantly from the target location.

For some applications, the wireless energy receiver coincides with or overlaps the target location.

For some applications, the one or more electrical light sources are selected from the group consisting of: respective light emitting diodes (LEDs), and respective laser diodes (LDs).

For some applications, the external controller is configured such that the wireless energy transmitter transmits energy at between 0.1 mW and 1 W.

For some applications, the external controller is configured such that the wireless energy transmitter transmits energy at an efficiency ratio of between 0.5% and 20%.

For some applications, the apparatus is configured such that the one or more electrical light sources would emit at least 0.005 lumens were the wireless energy transmitter to be disposed at 1 cm from the wireless energy receiver with a vacuum therebetween.

For some applications, the implant includes an electrical tissue stimulator.

For some applications, the electrical tissue stimulator is electrically coupled to the wireless energy receiver, and is configured to apply electrical stimulation to tissue of the subject when powered by the wireless energy receiver.

For some applications:

the wireless energy transmitter is a first wireless energy transmitter,

the wireless energy receiver is a first wireless energy receiver, which is configured to receive energy from the first wireless energy transmitter,

the external controller further includes a second wireless energy transmitter,

the implant further includes a second wireless energy receiver, which is configured to receive energy from the second wireless energy transmitter, and

the electrical tissue stimulator is electrically coupled to the second wireless energy receiver, and is configured to apply electrical stimulation to tissue of the subject when powered by the second wireless energy receiver.

For some applications, the implant further includes a rechargeable power source, which is coupled to the wireless energy receiver and the electrical tissue stimulator, and which is configured to recharge using energy received by the wireless energy receiver.

For some applications:

the wireless energy transmitter is a first wireless energy transmitter,

the wireless energy receiver is a first wireless energy receiver, which is configured to receive energy from the first wireless energy transmitter,

the external controller further includes a second wireless energy transmitter, and

the implant further includes:
a second wireless energy receiver, which is configured to receive energy from the second wireless energy transmitter; and
a rechargeable power source, which is coupled to the second wireless energy receiver and the electrical tissue stimulator, and which is configured to recharge using energy received by the second wireless energy receiver.

For some applications:

the implant further includes a reservoir, and

an external surface of the implant is shaped so as to define a fluid port, which is in fluid communication with the reservoir.

For some applications, the one or more electrical light sources are arranged so as to indicate a location of the fluid port on the implant.

For some applications, the one or more electrical light sources include at least three electrical light sources, which are arranged around the fluid port.

For some applications, the at least three electrical light sources are arranged equidistantly from the fluid port.

For some applications, the reservoir is a first reservoir, and the external controller includes a second reservoir.

For some applications, the external controller is shaped so as to define a Luer taper selected from the group of Luer tapers consisting of: a Luer-Lock and a Luer-Slip.

For some applications, the external controller further includes a needle, which is sized for insertion into the fluid port.

For some applications, the one or more electrical light sources are configured to emit visible light.

For some applications:

the external controller further includes an array of light detectors, at least one processor, and a user interface, and

the processor is configured to:
ascertain, responsively to one or more respective locations of the one or more electrical light sources, as detected by the array of light detectors, a desired movement of the external controller with respect to an external surface of skin of the subject, and
output, via the user interface, an indication of the desired movement.

For some applications, the one or more electrical light sources include at least three electrical light sources, and the at least three electrical light sources are disposed at vertices of an irregular polygon.

For some applications, the one or more electrical light sources are configured to emit infrared light.

There is further provided, in accordance with an application of the present invention, a method including:

placing an external controller, which includes a wireless energy transmitter, near an external surface of skin of a subject, in whose body an implant is implanted under the skin, which implant includes (a) a wireless energy receiver, which is configured to receive energy from the wireless energy transmitter, and (b) one or more electrical light sources, which are (i) electrically coupled to the wireless energy receiver, and (ii) configured to emit visible light upon being triggered by the wireless energy receiver receiving the energy from the wireless energy transmitter; and

moving the external controller responsively to the visible light emitted by the one or more electrical light sources.

For some applications, the one or more electrical light sources are arranged such that at least 10% of the light emitted from the one or more electrical light sources is emitted from an external surface of the implant.

For some applications, the one or more electrical light sources are arranged such that at least 25% of the light emitted from the one or more electrical light sources is emitted from the external surface of the implant.

For some applications, moving the external controller includes moving the external controller along the skin responsively to the visible light emitted by the one or more electrical light sources.

For some applications, moving the external controller responsively to the visible light emitted by the one or more electrical light sources includes moving the external controller toward a target location indicated by the one or more electrical light sources.

For some applications, moving the external controller toward the target location includes positioning the external controller within 5 mm of directly over the target location.

For some applications, the wireless energy receiver coincides with or overlaps the target location, and moving the external controller toward the target location includes positioning the external controller within 5 mm of directly over the wireless energy receiver.

For some applications, the one or more electrical light sources include a plurality of electrical light sources.

For some applications, the plurality of electrical light sources includes between two and four electrical light sources.

For some applications, the plurality of electrical light sources includes at least three electrical light sources.

For some applications, the at least three electrical light sources are disposed at vertices of a regular polygon.

For some applications, the at least three electrical light sources are arranged around a target location on the implant.

For some applications, the at least three electrical light sources are arranged equidistantly from the target location.

For some applications, the one or more electrical light sources are selected from the group consisting of: respective light emitting diodes (LEDs), and respective laser diodes (LDs).

For some applications, the external controller is configured such that the wireless energy transmitter transmits energy at between 0.1 mW and 1 W.

For some applications, the external controller is configured such that the wireless energy transmitter transmits energy at an efficiency ratio of between 0.5% and 20%.

For some applications, moving the external controller responsively to the visible light emitted by the one or more electrical light sources includes placing the external controller in a vicinity of the implant, by moving the external controller until the one or more electrical light sources begin to emit the visible light.

For some applications, moving the external controller responsively to the visible light emitted by the one or more electrical light sources further includes, after placing the external controller in the vicinity of the implant, moving the external controller toward a target location indicated by the one or more electrical light sources.

For some applications, placing the external controller in the vicinity of the implant includes placing the external controller within 10 mm of the implant.

For some applications, placing the external controller in the vicinity of the implant includes placing the external controller between 10 and 50 mm from the implant.

For some applications, moving the external controller includes moving the external controller responsively to an intensity of the visible light emitted by the one or more electrical light sources.

For some applications, the implant includes an electrical tissue stimulator.

For some applications, the electrical tissue stimulator is electrically coupled to the wireless energy receiver, and is configured to apply electrical stimulation to tissue of the subject when powered by the wireless energy receiver.

For some applications:

the wireless energy transmitter is a first wireless energy transmitter,

the wireless energy receiver is a first wireless energy receiver, which is configured to receive energy from the first wireless energy transmitter,

the external controller further includes a second wireless energy transmitter,

the implant further includes a second wireless energy receiver, which is configured to receive energy from the second wireless energy transmitter, and

the electrical tissue stimulator is electrically coupled to the second wireless energy receiver, and is configured to apply electrical stimulation to tissue of the subject when powered by the second wireless energy receiver.

For some applications, the implant further includes a rechargeable power source, which is coupled to the wireless energy receiver and the electrical tissue stimulator, and which is configured to recharge using energy received by the wireless energy receiver.

For some applications:

the wireless energy transmitter is a first wireless energy transmitter,

the wireless energy receiver is a first wireless energy receiver, which is configured to receive energy from the first wireless energy transmitter,

the external controller further includes a second wireless energy transmitter, and

the implant further includes:
a second wireless energy receiver, which is configured to receive energy from the second wireless energy transmitter; and
a rechargeable power source, which is coupled to the second wireless energy receiver and the electrical tissue stimulator, and which is configured to recharge using energy received by the second wireless energy receiver.

For some applications:

the implant further includes a reservoir,

an external surface of the implant is shaped so as to define a fluid port, which is in fluid communication with the reservoir, and

the method further includes, after moving the external controller, inserting a needle of the external controller through the skin and into the fluid port, and using the external controller to inject a liquid through the needle and the fluid port, and into the reservoir.

For some applications, the one or more electrical light sources are arranged so as to indicate a location of the fluid port on the implant.

For some applications, the one or more electrical light sources include at least three electrical light sources, which are arranged around the fluid port.

For some applications, the at least three electrical light sources are arranged equidistantly from the fluid port.

There is further provided, in accordance with an application of the present invention, a method including:

placing an external controller, which includes a wireless energy transmitter, near an external surface of skin of a subject, in whose body an implant is implanted under the skin, which implant includes (a) a wireless energy receiver, which is configured to receive energy from the wireless energy transmitter, and (b) one or more electrical light sources, which are (i) electrically coupled to the wireless energy receiver, and (ii) configured to emit light upon being triggered by the wireless energy receiver receiving the energy from the wireless energy transmitter, wherein the external controller further includes an array of light detectors, at least one processor, and a user interface, and wherein the processor is configured to: (1) ascertain, responsively to one or more respective locations of the one or more electrical light sources, as detected by the array of light detectors, a desired movement of the external controller with respect to an external surface of skin of the subject, and (2) output, via the user interface, an indication of the desired movement; and moving the external controller responsively to the indication of the desired movement.

For some applications, the one or more electrical light sources include at least three electrical light sources, and the at least three electrical light sources are disposed at vertices of an irregular polygon.

For some applications, the one or more electrical light sources are configured to emit infrared light.

The present invention will be more fully understood from the following detailed description of embodiments thereof, taken together with the drawings, in which:

### BRIEF DESCRIPTION OF THE DRAWINGS

FIG. 1 is a schematic illustration of an implant system, in accordance with an application of the present invention;

FIGS. 2A-B are schematic top-view illustrations of an implant of the implant system of FIG. 1, in accordance with respective applications of the present invention;

FIGS. 3A-B and 4A-B are schematic illustrations of respective configurations of the implant system of FIG. 1 in which an implant thereof comprises an electrical tissue stimulator, in accordance with respective applications of the present invention; and

FIG. 5 is a schematic illustration of another configuration of the implant system of FIG. 1 in which an implant thereof comprises a reservoir, in accordance with an application of the present invention.

### DETAILED DESCRIPTION OF APPLICATIONS

FIG. 1 is a schematic illustration of an implant system 520, in accordance with an application of the present invention. Implant system 520 comprises an implant 530, which is configured to be implanted in a body of a subject (such as under skin 554), and an external controller 540. External controller 540 comprises a wireless energy transmitter 542 (which typically comprises one or more coils), and implant 530 comprises a wireless energy receiver 544 (which typically comprises one or more coils), which is configured to receive energy from wireless energy transmitter 542. For example, the energy may be transmitted via inductive coupling or ultrasonically, as is known in the art. For some applications, external controller 540 is configured such that wireless energy transmitter 542 transmits energy at least 0.1 mW, no more than 1 W (e.g., no more than 100 mW), and/or between 0.1 mW and 1 W (e.g., between 0.1 mW and 100 mW). External controller 540 further comprises circuitry and, typically, a power source, such as a rechargeable power source, e.g., a battery. The figures are not drawn to scale; in particular, implant 530 is typically smaller in relation to external controller 540 than shown in the figures.

Implant 530 further comprises one or more electrical light sources 550, which are electrically coupled to wireless energy receiver 544, and are typically configured to emit visible light upon being triggered by wireless energy receiver 544 receiving the energy from wireless energy transmitter 542 (as a result, when wireless energy transmitter 542 is not near implant 530, the electrical light sources to not generate the light). The one or more light sources 550 are arranged to emit the visible light from an external surface 602 of implant 530, typically at a strength sufficient for the visible light to be easily visible through skin 554 by the eye (for example, when the implant is implanted 1 mm under skin 554). For example, in order to emit the visible light from external surface 602, the one or more light sources may be mounted on the external surface, or may be disposed inside implant 530 so as to emit the visible light through one or more optical windows on external surface 602 of implant 530. Typically, the one or more light sources 550 are arranged such that at least 10%, such as at least 25%, e.g., at least 50%, or at least 75% or at least 90% of the light emitted by the light sources is emitted from external surface 602 of implant 530.

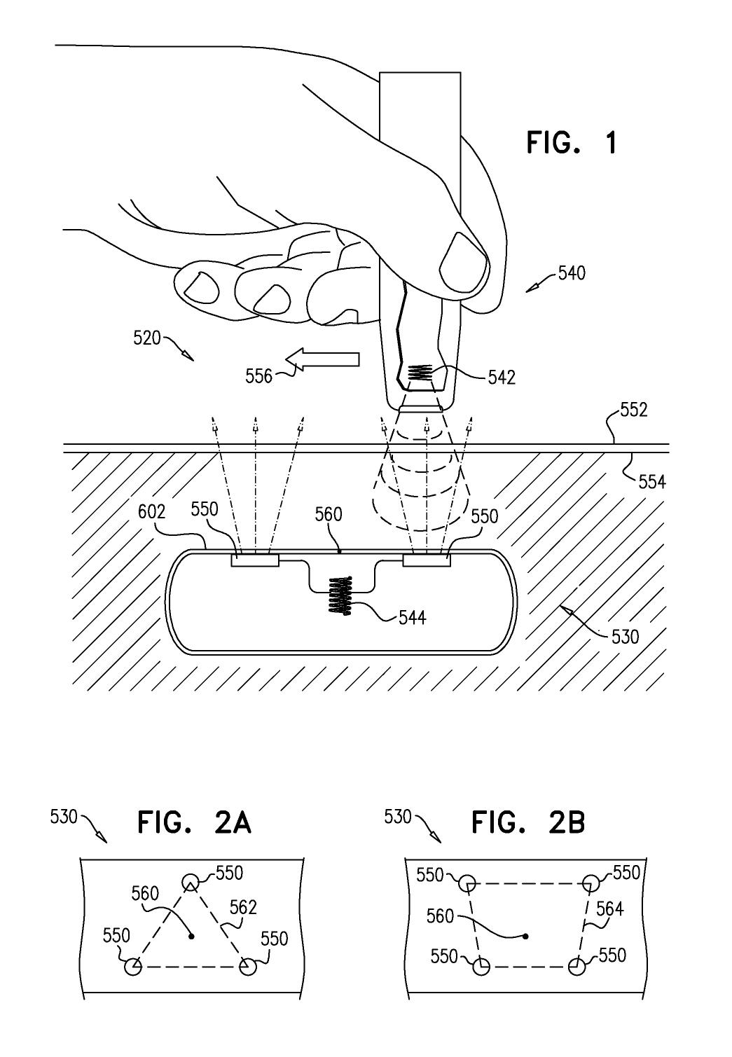

Typically, the one or more light sources 550 are configured to emit the visible light at one or more wavelengths that have good penetration through tissue, e.g., through skin 554. For example, the one or more light sources 550 may emit the visible light in the red or far-red portions of the spectrum, e.g., having peak emission at wavelength greater than 600 nm, such as greater than 650 nm. For some applications, such as described hereinbelow with reference to FIG. 2B, the emitted light is not visible, in which case the one or more light sources 550 may be configured to emit the non-visible light at one or more wavelengths that have good penetration through tissue, e.g., through skin 554, such at infrared portions of the spectrum, e.g., having peak emission at greater than 700 nm.

The one or more light sources are typically arranged so as to indicate a target location 560 on implant 530, which optionally coincides with or overlaps wireless energy receiver 544 of implant 530. A user places external controller 540 near an external surface 552 of skin 554 of the subject. The user places external controller 540 in a vicinity of implant 530 (e.g., within mm of implant 530, or between 10 and 50 mm from implant 530), which is implanted in the body under skin 554, by moving external controller 540 until the one or more light sources begin to emit the visible light. Typically, the user then positions external controller 540 directly over, or nearly directly over (e.g., within 5 mm of directly over), target location 560 by moving external controller 540 responsively to the visible light emitted by the one or more light sources 550 (the movement is schematically illustrated by way of example by an arrow 556). The one or more light sources 550 thus guide the user to move external controller 540 toward target location 560, and/or toward the implant itself if external controller 540 is not initially positioned over any portion of the implant. Typically, implant system 520 is configured such that the one or more light sources 550 would emit the visible light were wireless energy transmitter 542 to be disposed with a certain distance from wireless energy receiver 544 with a vacuum therebetween, such as within 10 mm of wireless energy receiver 544, or between 10 and 50 mm from wireless energy receiver 544. (It is to be understood that implant system 520 is actually not used in a vacuum with wireless energy transmitter 542 disposed at these distances; these parameters instead specify conditions under which the one or more light sources would emit the visible light.)

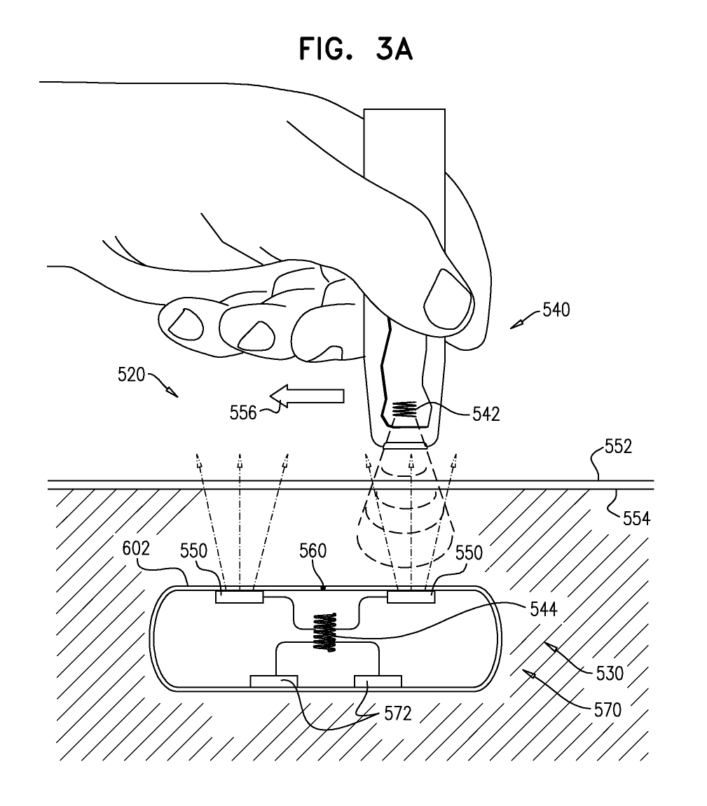

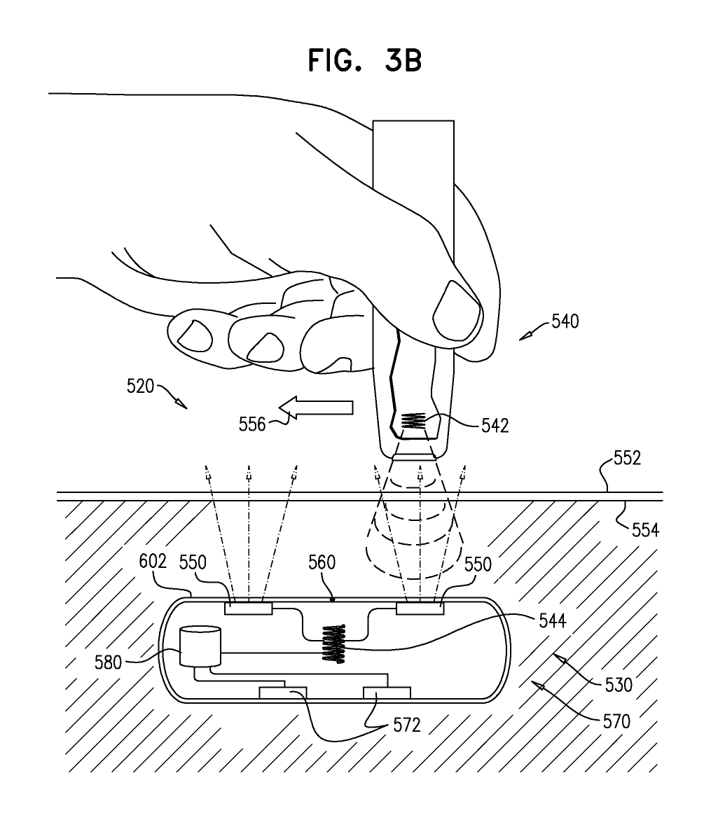

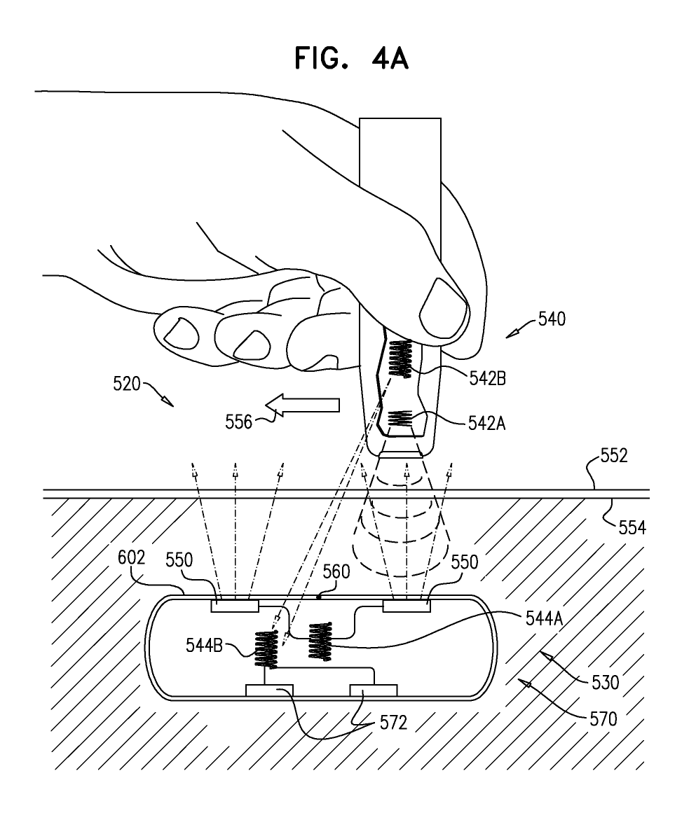

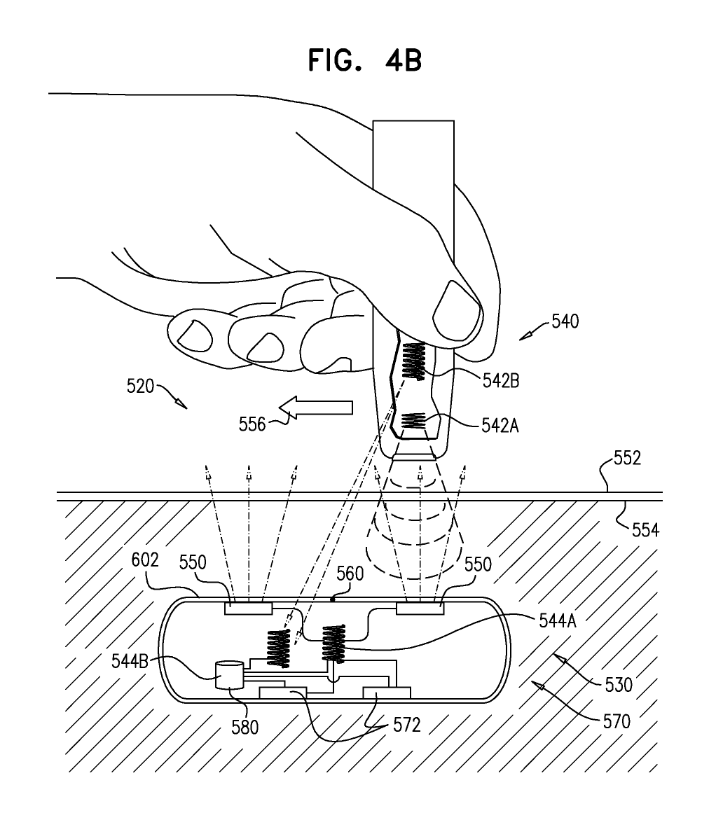

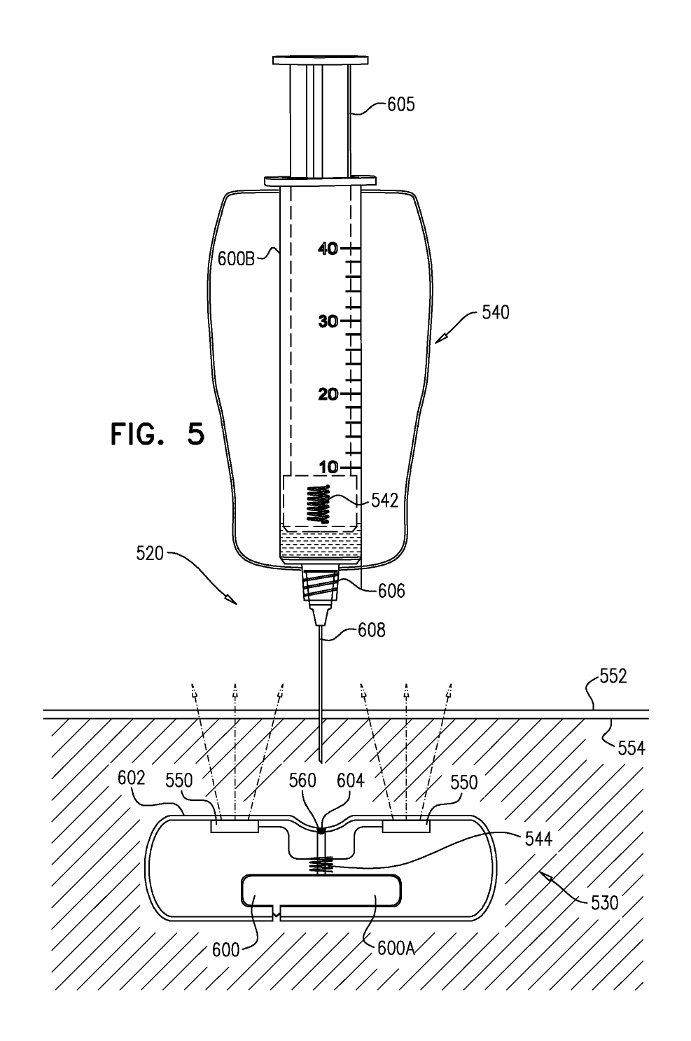

Such accurate positioning may, for example, aid in transmitting energy from external controller 540 to implant 530, such as described hereinbelow with reference to FIGS. 3A-B and 4A-B, and/or in enabling the user to fill a reservoir of implant 530 from external controller 540, such as described hereinbelow with reference to FIG. 5. Alternatively or additionally, such accurate positioning may enable lower-energy transmission of data to implant 530 from external controller 540, and/or from implant 530 to external controller 540. The user may be the subject or somebody else assisting the user, such as a healthcare worker or relative.

Typically, the user moves external controller 540 along skin 554 responsively to the visible light emitted by the one or more light sources 550. For some applications, the user aims external controller 540 toward target location 560 and/or toward implant 530 by moving external controller 540 responsively to an intensity of the visible light emitted by the one or more light sources 550. For example, the amount of power transmitted may increase as wireless energy transmitter 542 (and thus external controller 540) is closer to wireless energy receiver 544, resulting in an increase the intensity of the emitted visible light. For some applications, wireless energy receiver 544 overlaps target location 560 (as shown). For some applications, the entire implant 530 is the target.

Reference is still made to FIG. 1, and is additionally made to FIGS. 2A-B, which are schematic top-view illustrations of implant 530, in accordance with respective applications of the present invention. For some applications, the one or more light sources 550 comprise a plurality of light sources 550, for example, between two and four light sources 550, or at least three light sources 550. Alternatively, the one or more light sources 550 comprise exactly one light source 550.

For some applications, such as shown in FIG. 2A, in order to aid the user's identification of target location 560 on implant 530, the at least three light sources 550 are disposed at vertices of a regular polygon, such as a triangle 562 (as shown) or a square (not shown), and/or are arranged around target location 560. Optionally, the at least three light sources 550 are arranged equidistantly from target location 560 (as shown).

In some applications of the present invention, external controller 540 further comprises an array of light detectors (e.g., a CCD sensor), at least one processor, and a user interface, which typically comprises a graphical display, other visual outputs, and/or an audio generator. The user interface may be incorporated into a housing of the external controller 540, or may be in a separate component in data communication (wireless or wired) with the housing of external controller 540. The processor is configured to (a) ascertain, responsively to one or more respective locations of the one or more light sources 550 (as detected by the array of light detectors), a desired movement of external controller 540 with respect to an external surface of skin 554, such as by using a pattern-recognition algorithm, and (b) output, via the user interface, an indication of the desired movement. A user moves external controller 540 in order to better align the external controller with the implantable unit (typically with target location 560). For some applications, the indication of the desired movement includes a direction of the desired movement with respect to the external surface of the skin. For some applications, the user interface is configured to guide the user to position the external controller at the most appropriate location, e.g., using crosshairs, arrows, or other visual or audio indicators (e.g., including pitch and/or volume) of the desired location and/or desired motion toward the desired location.

In these applications, the light generated by the one or more light sources 550 may be either visible or not visible, as described hereinabove with reference to FIG. 1.

For some applications, such as shown in FIG. 2B, in order to aid identification of target location 560 on implant 530 by the processor of external controller 540, the at least three light sources 550 are disposed at vertices of an irregular polygon (i.e., three or more points in a non-symmetrical arrangement), such as a four-sided irregular polygon 564 (as shown) or a non-equilateral triangle, e.g., a scalene triangle (not shown). The use of an irregular polygon may facilitate identification by the processor of external controller 540 of the directionality and rotational alignment of the implant with respect to external controller 540, such as by using a pattern-recognition algorithm. Alternatively or additionally, implant 530 comprises exactly two light sources 550, which optionally generate respective light signals having respective different properties, such as different timing patterns or wavelengths, which are detected by external controller 540. Optionally, different light sources 550 may have different emission spectra and, optionally, the detection system in external controller 540 may differentiate between the different spectra, e.g., using different colored filters placed in front of different detectors.

Reference is made to FIGS. 1 and 2A-B. For some applications, the one or more light sources 550 comprise one or more respective light emitting diodes (LEDs), or one or more respective laser diodes (LDs). For some applications, external controller 540 is configured such that wireless energy transmitter 542 transmits energy at an efficiency ratio of between 0.5% and 20%. For some applications, implant system 520 is configured such that the one or more light sources 550 would emit at least 0.005 lumens were wireless energy transmitter 542 to be disposed at 1 cm from wireless energy receiver 544 with a vacuum therebetween. (It is to be understood that implant system 520 is actually not used in a vacuum with wireless energy transmitter 542 disposed at 1 cm from wireless energy receiver 544; these parameters instead specify conditions under which the described luminous flux is to be measured.)

Reference is now made to FIGS. 3A-B and 4A-B, which are schematic illustrations of respective configurations of implant system 520 in which implant 530 comprises an electrical tissue stimulator 570, in accordance with respective applications of the present invention. For example, electrical tissue stimulator 570 may be configured to apply electrical stimulation to nervous tissue (e.g., to the vagus nerve for treating epilepsy or heart failure, to the tibial nerve for treating pain, or to the pudendal nerve for treating erectile dysfunction), cardiac tissue, and/or muscle tissue. Electrical tissue stimulator 570 typically comprises one or more electrodes 572. For some applications, electrical tissue stimulator 570 is configured to be injectable, as is known in the art.

For some applications, such as shown in FIG. 3A, electrical tissue stimulator 570 is electrically coupled to wireless energy receiver 544, and is configured to apply electrical stimulation to tissue of the subject when powered by wireless energy receiver 544. Thus, in this configuration, the energy received by wireless energy receiver 544 (which is typically transmitted by wireless energy transmitter 542) powers both the one or more light sources 550 and electrical tissue stimulator 570. Alternatively, for some applications, such as shown in FIG. 3B, implant 530 further comprises a rechargeable power source 580 (e.g., a battery and/or a capacitor), which is coupled to wireless energy receiver 544 and electrical tissue stimulator 570, and which is configured to recharge (i.e., become charged) using energy received by wireless energy receiver 544. Optionally, electrical tissue stimulator 570 further comprises circuitry for controlling the tissue stimulation, as is known in the art. For these applications illustrated in FIGS. 3A-B, wireless energy receiver 544 typically coincides with or overlaps target location 560.

For some applications, such as shown in FIG. 4A-B, wireless energy transmitter 542 is a first wireless energy transmitter 542A, and wireless energy receiver 544 is a first wireless energy receiver 544A, which is configured to receive energy from first wireless energy transmitter 542A. External controller 540 further comprises a second wireless energy transmitter 542B, and implant 530 further comprises a second wireless energy receiver 544B, which is configured to receive energy from second wireless energy transmitter 542B. For some applications, such as shown in FIG. 4A, electrical tissue stimulator 570 is electrically coupled to second wireless energy receiver 544B, and is configured to apply electrical stimulation to tissue of the subject when powered by second wireless energy receiver 544B. For other applications, such as shown in FIG. 4B, implant 530 further comprises rechargeable power source 580, which is coupled to second wireless energy receiver 544B and electrical tissue stimulator 570, and which is configured to recharge (i.e., become charged) using energy received by second wireless energy receiver 544B.

Reference is now made to FIG. 5, which is a schematic illustration of another configuration of implant system 520 in which implant 530 comprises a reservoir 600, such as a drug reservoir, in accordance with an application of the present invention. An external surface 602 of implant 530 is shaped so as to define a fluid port 604, which is in fluid communication with reservoir 600. Fluid port 604 coincides with or overlaps target location 560. The one or more light sources 550 guide the user to move external controller 540 toward fluid port 604, typically in order to transdermally fill reservoir 600. For example, reservoir 600 may need to be filled with drug occasionally and/or periodically.

Typically, reservoir 600 is a first reservoir 600A, and external controller 540 comprises a second reservoir 600B (such as a second drug reservoir), which may, for example, be provided by a conventional syringe 605 (as shown) or any other container. For some applications, external controller 540 (e.g., syringe 605) is shaped so as to define a Luer taper 606 selected from the group of Luer tapers consisting of: a Luer-Lock (as shown) and a Luer-Slip (not shown). For some applications, external controller 540 further comprises a needle 608, which is configured to be coupled to the Luer taper, as is known in the art, and which is sized for insertion into fluid port 604. After positioning needle 608 over fluid port 604 at target location 560, the user inserts needle 608 of external controller 540 through skin 554 and into fluid port 604, and typically uses external controller 540 to inject a liquid, such as a drug, through needle 608 and fluid port 604, and into first reservoir 600A.

For example, fluid port 604 may comprise a self-sealing membrane, as is known in the art. Alternatively, the user uses needle 608 to withdraw fluid from first reservoir 600A, such as body fluids that have accumulated in first reservoir 600A.

Typically, the one or more light sources 550 are arranged so as to indicate a location of fluid port 604. For some applications, the one or more light sources 550 comprise at least three light sources 550, which are arranged around fluid port 604. For example, the at least three light sources 550 may be arranged such as described hereinabove with reference to FIGS. 2A-B.

For some applications, implant 530 comprises both electrical tissue stimulator 570 and reservoir 600, and the techniques described hereinabove with reference to (a) FIGS. 3A-4B and (b) FIG. 5 are used in combination (configuration not shown). For example, implant 530 may have two target locations 560, a first one indicating the location of wireless energy receiver 544, and a second one indicating the location of fluid port 604. For example, the arrangement of light sources described hereinabove with reference to FIG. 2B may be used to aid the identification of the target locations by the processor of external controller 540, in order to direct the user to reposition the external controller twice, once for each of the two target locations.

Reference is made to FIGS. 1-5. For some applications, implant 530 comprises a vibrating element, which is configured to vibrate when wireless energy transmitter 542 is moved near to wireless energy receiver 544. For some applications, implant 530 also comprises the one or more light sources 550, while for other applications, the implant does not comprise the one or more light sources in this configuration. For some applications, the vibrating element is configured to provide haptic vibration (like a cellular telephone ring). For other applications, the vibrating element is configured to provide audible vibration as sound waves. For some applications, the user aims by moving external controller 540 responsively to an intensity of the vibration (either haptic or audible). For example, the amount of power transmitted may increase as wireless energy transmitter 542 (and thus external controller 540) is closer to wireless energy receiver 544, resulting in an increase the intensity of the vibration.

Reference is again made to FIGS. 1-5. For some application, illumination of the one or more light sources 550 aids in precise locating implant 530 for explantation of the implant.

It will be appreciated by persons skilled in the art that the present invention is not limited to what has been particularly shown and described hereinabove. Rather, the scope of the present invention includes both combinations and subcombinations of the various features described hereinabove, as well as variations and modifications thereof that are not in the prior art, which would occur to persons skilled in the art upon reading the foregoing description.

1. Apparatus comprising:
an external controller, which comprises a wireless energy transmitter; and
an implant, which is configured to be implanted in a body of a subject, and which comprises:
a wireless energy receiver, which is configured to receive energy from the wireless energy transmitter; and
one or more electrical light sources, which are (a) electrically coupled to the wireless energy receiver, and (b) configured to emit light upon being triggered by the wireless energy receiver receiving the energy from the wireless energy transmitter.

2. The apparatus according to claim 1, wherein the one or more electrical light sources are arranged such that at least 10% of the light emitted from the one or more electrical light sources is emitted from an external surface of the implant.

3. (canceled)

4. The apparatus according to claim 1, wherein the one or more electrical light sources are arranged so as to indicate a location of a target location on the implant.

5. The apparatus according to claim 4, wherein the wireless energy receiver coincides with or overlaps the target location.

6. The apparatus according to claim 1, wherein the one or more electrical light sources comprise a plurality of electrical light sources.

7-9. (canceled)

10. The apparatus according to claim 6, wherein the plurality of electrical light sources comprises at least three electrical light sources that are arranged around a target location on the implant.

11. The apparatus according to claim 10, wherein the at least three electrical light sources are arranged equidistantly from the target location.

12-16. (canceled)

17. The apparatus according to claim 1, wherein the implant comprises an electrical tissue stimulator.

18-21. (canceled)

22. The apparatus according to claim 1,
wherein the implant further comprises a reservoir, and
wherein an external surface of the implant is shaped so as to define a fluid port, which is in fluid communication with the reservoir.

23. The apparatus according to claim 22, wherein the one or more electrical light sources are arranged so as to indicate a location of the fluid port on the implant.

24-28. (canceled)

29. The apparatus according to claim 1, wherein the one or more electrical light sources are configured to emit visible light.

30. The apparatus according to claim 1,
wherein the external controller further comprises an array of light detectors, at least one processor, and a user interface, and
wherein the processor is configured to:
ascertain, responsively to one or more respective locations of the one or more electrical light sources, as detected by the array of light detectors, a desired movement of the external controller with respect to an external surface of skin of the subject, and
output, via the user interface, an indication of the desired movement.

31-32. (canceled)

33. A method comprising:
placing an external controller, which comprises a wireless energy transmitter, near an external surface of skin of a subject, in whose body an implant is implanted under the skin, which implant comprises (a) a wireless energy receiver, which is configured to receive energy from the wireless energy transmitter, and (b) one or more electrical light sources, which are (i) electrically coupled to the wireless energy receiver, and (ii) configured to emit visible light upon being triggered by the wireless energy receiver receiving the energy from the wireless energy transmitter; and
moving the external controller responsively to the visible light emitted by the one or more electrical light sources.

34. The method according to claim 33, wherein the one or more electrical light sources are arranged such that at least 10% of the light emitted from the one or more electrical light sources is emitted from an external surface of the implant.

35. (canceled)

36. The method according to claim 33, wherein moving the external controller comprises moving the external controller along the skin responsively to the visible light emitted by the one or more electrical light sources.

37. The method according to claim 33, wherein moving the external controller responsively to the visible light emitted by the one or more electrical light sources comprises moving the external controller toward a target location indicated by the one or more electrical light sources.

38. The method according to claim 37, wherein moving the external controller toward the target location comprises positioning the external controller within 5 mm of directly over the target location.

39. (canceled)

40. The method according to claim 33, wherein the one or more electrical light sources comprise a plurality of electrical light sources.

41-43. (canceled)

44. The method according to claim 40, wherein the plurality of electrical light sources comprises at least three electrical light sources that are arranged around a target location on the implant.

45. The method according to claim 44, wherein the at least three electrical light sources are arranged equidistantly from the target location.

46-48. (canceled)

49. The method according to claim 33, wherein moving the external controller responsively to the visible light emitted by the one or more electrical light sources comprises placing the external controller in a vicinity of the implant, by moving the external controller until the one or more electrical light sources begin to emit the visible light.

50. The method according to claim 49, wherein moving the external controller responsively to the visible light emitted by the one or more electrical light sources further comprises, after placing the external controller in the vicinity of the implant, moving the external controller toward a target location indicated by the one or more electrical light sources.

51-53. (canceled)

54. The method according to claim 33 any one of claims 33 53, wherein the implant comprises an electrical tissue stimulator.

55-58. (canceled)

59. The method according to claim 33 any one of claims 33 53,
wherein the implant further comprises a reservoir,
wherein an external surface of the implant is shaped so as to define a fluid port, which is in fluid communication with the reservoir, and
wherein the method further comprises, after moving the external controller, inserting a needle of the external controller through the skin and into the fluid port, and using the external controller to inject a liquid through the needle and the fluid port, and into the reservoir.

60. The method according to claim 59, wherein the one or more electrical light sources are arranged so as to indicate a location of the fluid port on the implant.

61-62. (canceled)

63. A method comprising:
placing an external controller, which comprises a wireless energy transmitter, near an external surface of skin of a subject, in whose body an implant is implanted under the skin, which implant comprises (a) a wireless energy receiver, which is configured to receive energy from the wireless energy transmitter, and (b) one or more electrical light sources, which are (i) electrically coupled to the wireless energy receiver, and (ii) configured to emit light upon being triggered by the wireless energy receiver receiving the energy from the wireless energy transmitter, wherein the external controller further comprises an array of light detectors, at least one processor, and a user interface, and wherein the processor is configured to: (1) ascertain, responsively to one or more respective locations of the one or more electrical light sources, as detected by the array of light detectors, a desired movement of the external controller with respect to an external surface of skin of the subject, and (2) output, via the user interface, an indication of the desired movement; and
moving the external controller responsively to the indication of the desired movement.

64-65. (canceled)
